(19) United States
(12) Patent Application Publication
(10) Pub. No.: US 2022/0211681 A1
(43) Pub. Date: Jul. 7, 2022

(54) COMPOSITIONS AND METHODS FOR PREVENTING INFLAMMATORY CONDITIONS

(71) Applicant: Trustees of Tufts College, Medford, MA (US)

(72) Inventors: Joel Mason, Boston, MA (US); Xian Wu, Boston, MA (US); Jimmy W. Crott, Boston, MA (US)

(21) Appl. No.: 17/611,730

(22) PCT Filed: May 15, 2020
(86) PCT No.: PCT/US2020/033206

§ 371 Date: Nov. 16, 2021

Related U.S. Application Data
(60) Provisional application No. 62/849,373, filed on May 17, 2019.

(51) Int. Cl. A61K 31/4415; A61K 31/122

(52) U.S. Cl. CPC A61K 31/4415; A61K 31/122

(57) ABSTRACT

Provided herein are compositions and methods for preventing and/or reducing the risk of inflammatory disease in subject. In particular, provided herein are methods for preventing colorectal cancer in a subject by administering to the subject one or more curcuminoids and vitamin B6.

The present application claims priority to U.S. Provisional Patent Application Ser. No. 62/849,373, filed May 17, 2019, the disclosure of which is herein incorporated by reference in its entirety.

### STATEMENT REGARDING FEDERALLY SPONSORED RESEARCH OR DEVELOPMENT

This invention was made with government support under agreement 2016-67017-24580 awarded by the United. States Department of Agriculture. The government has certain rights in the invention.

### FIELD

Provided herein are compositions and methods for preventing and/or reducing the risk of inflammatory conditions, such as colorectal cancer. In particular, provided herein are compositions comprising curcumin and vitamin B6 for use in preventing colorectal cancer.

### BACKGROUND

Colorectal cancer (CRC) is the third most common cancer worldwide and therefore represents a major public health issue globally. In the U.S. alone, over 145,000 new cases will be diagnosed in in 2019, and over 51,000 individuals will die from the disease. CRC is the third most common cancer in each gender and the second most common cause of cancer death in the U.S. Epidemiological studies have demonstrated that obesity is a prominent risk factor for CRC in bath sexes, with a 1.5- to 2-fold increase in men, and a 1.2- to 1.5-fold increase in women. Low-grade, chronic inflammation in the colons produced by obesity may be an essential factor in mediating the pro-carcinogenic effects of obesity in colorectal tumorigenesis.

The increasing adoption of screening colonoscopies over the past three decades led to a sizeable decline in the incidence and mortality from the disease, establishing the value of preventive strategies. However, colonoscopy is an invasive procedure that carries with it well-defined risks. Also, the quality of the test varies: studies show that 2%-12% of colonoscopies miss large pre-cancerous polyps. Further, the cost of providing periodic screening colonoscopies for the entire U.S. adult population imposes an enormous financial burden on the health care system. As such, improved techniques for preventing colorectal cancer, including colorectal cancer in overweight and obese individuals, are needed.

### SUMMARY

Provided herein are compositions and methods for preventing and/or reducing the risk of inflammatory disease in a subject. In particular, provided herein are compositions and methods for preventing and/or reducing the risk of colorectal cancer.

For example, in some embodiments, provided herein is a method preventing and/or reducing the risk of colorectal cancer, comprising administering to the subject one or more curcuminoids and vitamin B6. The one or more curcuminoids and vitamin B6 may be provided to the subject jointly in a single composition. The one or more curcuminoids and vitamin B6 may be provided to the subject separately in two or more compositions or in a single composition (e.g., pharmaceutical composition, nutritional supplement, or food product). In some embodiments, the administering prevents the formation of pre-cancerous (adenomatous) polyps and/or or malignant neoplasms.

The present disclosure is not limited to particular formulations of curcuminoids. In some embodiments, the curcuminoids are one or more of curcumin, demethoxycurcumin, bisdemethoxycurcumin, or combinations thereof. For example, in some embodiments, the one or more curcuminoids comprise 1-100% curcumin, 0-99% demethoxycurcumin, and 0-99% bisdemethoxycurcumin, 60-100% curcumin, 1-50% demethoxycurcumin, and 1-30% bisdemethoxycurcurnin, 70-90% curcumin, 10-20% demethoxycurcumin, and 1-10% bisdemethoxycurcumin or 79.5% curcumin, 17.9% demethoxycurcurnin and 2.8% bisdemethoxycurcumin.

The present disclosure is not limited to particular formulations of vitamin B6. In some embodiments, the vitamin B6 is provided as pyridoxine or as a salt thereof.

In some embodiments, the subject is at risk for colorectal cancer (e.g., as result of a clinical finding selected from, for example, one or more of a family history of colorectal cancer, has previously had colorectal cancer, a finding of a polyp and/or precancerous lesion during colonoscopy or other diagnostic test, or a finding of a molecular marker associated with colorectal cancer). In some embodiments, the subject is overweight or obese. In some embodiments, the subject is not overweight or obese.

Further embodiments provide the use of a composition comprising one or more curcuminoids and vitamin B6 to prevent or reduce the risk of an inflammatory disease.

Yet other embodiments provide a composition comprising one or more curcuminoids and vitamin B6 (e.g., for use in preventing or reducing the risk of inflammatory disease in a subject).

Additional embodiments will be apparent to persons skilled in the relevant art based on the teachings contained herein.

### BRIEF DESCRIPTION OF THE DRAWINGS

FIG. 1A-FIG. 1B shows the experimental design (FIG. 1A) and weekly body weights obtained over the course of the study (FIG. 1B).

FIG. 2 shows the effects of CM B6 and CUR/B6 co-treatment on level of IL-6 in plasma (n=10) and calprotectin in feces (n=20) of FVB mice. Frozen fecal samples obtained in the colon upon euthanasia and then measured by Mouse S100A8/S100A9 Heterodimer DuoSet ELISA according to manufacturer's instructions (R&D Systems, Minneapolis, Minn.) and normalized by protein concentrations. Data are shown as the mean±SE, Different superscript letters in the bar charts indicate significant differences (p<0.05) by one-way analysis of variance (ANOVA) with Tukey's test.

FIG. 3 shows the effects of CUR, B6 and CUR/B6 co-treatment on expression levels of p-PI3K and PI3K in the colonic mucosa, and p65, p-p65 and β-catenin in the nuclear fraction of the colonic mucosa. Each lane contained colonic mucosa collected from two individual mice that were pooled for immunoblot analysis (n=6). Data are representative of two independent experiments. Band intensity was normalized to Lamin B1 (nuclear fraction) or β-actin (whole tissue lysate) and measured by Quantity One 1-D Analysis Software (Bio-Rad). Data are shown as the mean±SE. Multiple comparisons with Hochberg method were employed to control the False Discovery Rate. Different superscript letters in the bar charts indicate significant differences (p<0.05) by one-way analysis of variance (ANOVA) with Tukey's test.

### DEFINITIONS

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “co-administration” and variations thereof refer to the administration of at least two agent(s) or therapies to a subject (e.g., a composition disclosed herein and one or more therapeutic agents). In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

The term “colorectal cancer” refers to a malignant neoplasm of the large intestine/colon within a given subject, wherein the neoplasm is of epithelial origin and is also referred to as a carcinoma of the large intestine/colon, According to the disclosure, colorectal cancer is defined according to its type, stage and/or grade. Typical staging systems known to those skilled in the art. The term “colorectal cancer”, when used without qualification, includes both localized and metastasised colorectal cancer. The term “colorectal cancer” can be qualified by the terms “localized” or “metastasised” to differentiate between different types of tumor as those words are defined herein. The terms “colorectal cancer” and “malignant disease of the large intestine/colon” are used interchangeably herein. The term “colorectal cancer” includes, but is not limited to, colon cancer, rectal cancer, and bowel cancer.

The term “curcuminoid” or “CUR” as used interchangeably herein refers to any one or more polyphenolic pigments found within turmeric. Curcuminoids are a family of active compounds including curcumin, demethoxycurcumin, and bisdemethoxycurcumin. The term curcuminoid may refer to any one or more of curcumin, demethoxycurcumin, and bisdemethoxycurcumin.

The term “diagnostic assay” can be used interchangeably with “diagnostic method” and refers to the detection of the presence or nature of a pathologic condition.

The terms “neoplasm” or “tumor” may be used interchangeably and refer to an abnormal mass of tissue wherein growth of the mass surpasses and is not coordinated with the growth of normal tissue. A neoplasm or tumor may be defined as “benign” or “malignant” depending on the following characteristics: degree of cellular differentiation including morphology and functionality, rate of growth, local invasion and metastasis. A “benign” neoplasm is generally well differentiated, has characteristically slower growth than a malignant neoplasm and remains localized to the site of origin. In addition, a benign neoplasm does not have the capacity to infiltrate, invade or metastasize to distant sites. A “malignant” neoplasm is generally poorly differentiated (anaplasia), has characteristically rapid growth accompanied by progressive infiltration, invasion and destruction of the surrounding tissue. Furthermore, a malignant neoplasm has to capacity to metastasize to distant sites. In the colorectum a benign neoplasm is often referred to as an adenomatous polyp, or adenoma.

The term “metastasis” refers to the spread or migration of cancerous cells from a primary (original) tumor to another organ or tissue, and is typically identifiable by the presence of a “secondary tumor” or “secondary cell mass” of the tissue type of the primary (original) tumor and not of that of the organ or tissue in which the secondary (metastatic) tumor is located. For example, a colorectal cancer that has migrated to bone is said to be metastasised colorectal cancer, and consists of cancerous colorectal cancer cells in the large intestine/colon as well as cancerous colorectal cancer cells growing in bone tissue.

As used herein, the terms “prevent,” “prevention,” and preventing” may refer to reducing the likelihood of a particular condition or disease state (e.g., colorectal cancer) from occurring in a subject not presently experiencing or afflicted with the condition or disease state. The terms do not necessarily indicate complete or absolute prevention. For example “preventing colorectal cancer” refers to reducing the likelihood of colorectal cancer occurring in a subject not presently experiencing or diagnosed with colorectal cancer. The terms may also refer to delaying the onset of a particular condition or disease state (e.g., colorectal cancer) in a subject not presently experiencing or afflicted with the condition or disease state. In order to “prevent colorectal cancer” a composition or method need only reduce the likelihood and/or delay the onset of colorectal cancer, not completely block any possibility thereof “Prevention,” encompasses any administration or application of a therapeutic or technique to reduce the likelihood or delay the onset of a disease developing (e.g., in a mammal, including a human). Such a likelihood may be assessed for a population or for an individual.

As used herein, the terms “treat,” “treatment,” and “treating” refer to reducing the amount or severity of a particular condition, disease state (e.g., colorectal cancer), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the term “subject” as used herein includes all members of the animal kingdom including mammals, and suitably refers to humans. Optionally, the term “subject” includes mammals that have been diagnosed with a colorectal cancer or are in remission.

### DETAILED DESCRIPTION

In one aspect, provided herein provided herein are compositions and methods of preventing an inflammatory disease in a subject. The methods of preventing an inflammatory disease in a subject comprise providing to the subject one or more curcuminoids and vitamin B6. The one or more curcuminoids and the vitamin B6 may be provided to the subject jointly in a single composition. Alternatively, the one or more curcuminoids and the vitamin B6 may be provided to the subject separately in two or more compositions. For example, the one or more curcuminoids may be provided to the subject in one or more compositions and the vitamin B6 may be provided to the subject in a separate composition.

The curcuminoids may be selected from curcumin, demethoxycurcumin, bisdemethoxycurcumin, and combinations thereof. In some embodiments, one or more curcuminoids may comprise curcumin alone. In other embodiments, the one or more curcuminoids may comprise curcumin and an additional curcuminoid. For example, the curcuminoids may comprise curcumin and demethoxycurcumin, curcumin and bisdemethoxycurcumin, or curcumin and demethoxycurcumin and bisdemethoxycurcumin. In some embodiments, the method comprises providing to the subject a single composition comprising curcumin, demethoxycurcumin, bismedothyxycurcumin, and vitamin B6.

The one or more curcuminoids may be provided at any suitable ratio. In some embodiments, the one or more curcuminoids may comprise 1-100% curcumin, 0-99% demethoxycurcumin, and 0-99% bisdemethoxycurcumin. For example, the one or more curcuminoids may comprise 1-100% curcumin, 10-90% curcumin; curcumin, 30-70% curcumin. 40-60% curcumin, or 50% curcumin; 0-99% demethoxycurcumin, 10-90% demethoxycurcumin, 20-80% demethoxycurcumin, 30-70% demethoxycurcumin, 40-60% demethoxycurcumin, or 50% demethoxycurcumin; and 0-99% bisdemethoxycurcumin, 10-90% bisdemethoxycurcumin, 20-80% bisdemethoxycurcumin, 30-70% bisdemethoxycurcumin, 40-60% bisdemethoxycurcumin, or 50% bisdemethoxycurcumin.

In particular embodiments, the one or more curcuminoids may comprise 1-100% curcumin, 0-30% demethoxycurcumin, and 0-10% bisdemethoxycurcumin. For example, the one or more curcuminoids may comprise 60-100% curcumin, 1-50% demethoxycurcumin, and 1-30% bisdemethoxycurcumin. In particular embodiments, the one or more curcuminoids comprise 70-90% curcumin, 10-20% demethoxycurcumin, and 1-10% bisdemethoxycurcumin. For example, the one or more curcuminoids may be provided as Curcumin C3 Complex, a blend of 79.5% curcumin, 17.9% demethoxycurcumin and 2,8% bisdemethoxycurcumin (Sabinsa Corporation, East Windsor, N.J., USA), described in U.S. Pat. No. 5,861,415 herein incorporated by reference in its entirety. Other commercially available curcuminoids or curcuminoid blends may be used. Alternatively, the one or more curcuminoids may be prepared in a customized ratio prior to use in the subject.

The one or more curcuminoids and the vitamin B6 may be provided to the subject in any suitable amount. Suitable amounts may vary depending on factors known in the art including age of the subject, weight of the subject, frequency of administration, form of administration, other medications being administered to the subject, and the like.

In some embodiments; the one or more curcuminoids may be administered to the subject at a dose equivalent to 0.1% to 0.5% of the body weight of the subject per day. In particular embodiments, the one or more curcuminoids may be provided to the subject at a dose equivalent to about 0.2% of the body weight of the subject per day.

For example, the one or more curcuminoids may be provided to the subject at a dose of about 100 mg to about 5 g per day. For example, the one or more curcuminoids may be provided to the subject at a dose of about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1 g, about 1.5 g, about 2 g, about 2.5 g, about 3 g, about 3.5 g, about 4 g, about 4.5 g, or about 5 g per day.

In some embodiments, the vitamin B6 may be provided to the subject at a dose of about 100 μg to about 20 mg per day. For example, the vitamin B6 may be administered to the subject at a dose of about 100 μg, about 200 μg, about 300 μg, about 400 μg, about 500 μg, about 600 μg, about 700 μg, about 800 about 900 μg, about 1 mg, about 1.5 mg, about 2 mg, about 2.5 mg, about 3 mg, about 3.5 mg, about 4 mg, about 4.5 mg, about 5 mg, about 7.5 mg, about 10 mg, about 15 mg, or about 20 mg per day.

The vitamin B6 may be provided as any suitable form of vitamin B6. For example, the vitamin B6 may be provided as one or more of pyridoxine, pyridoxamine, pyridoxal, or salts thereof. For example, the vitamin B6 may be provided as pyridoxine. In particular embodiments, the vitamin B6 may be provided as pyridoxine hydrochloride. Any suitable source of vitamin B6 may be utilized (e.g., commercial sources).

The one or more curcuminoids and the vitamin B6 may be provided to the subject in any suitable dosage form. For example, the one or more curcuminoids may be provided to the subject in one or more capsules, extracts, pills, food products, supplements, or the like. In some embodiments, the one or more curcuminoids and the vitamin B6 are separately microencapsulated.

In some embodiments, the one or more curcuminoids and the vitamin B6 are provided to the subject in a food or beverage product. For example, the one or more curcuminoids and the vitamin B6 may be provided to the subject in a food product or in a beverage product. In some embodiments, the one or more curcuminoids and the vitamin B6 are provided as a nutritional supplement (e.g., to be administered alone or added to a food or beverage product).

In some embodiments, the composition or compositions comprising the one or more curcuminoids and the vitamin B6 are administered alone, while in some other embodiments, the compositions further comprise one or more pharmaceutically acceptable carriers. Each carrier must be “acceptable” in the sense that it is compatible with the other ingredients of the formulation and not injurious to the subject. Suitable carriers depend on the intended route of administration to the subject. Contemplated routes of administration include those oral, rectal, nasal, topical (including transdermal, buccal and sublingual), vaginal, parenteral (including subcutaneous, intramuscular, intravenous and intradermal) and pulmonary administration. In some embodiments, the composition or compositions are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present disclosure suitable for oral administration may be presented as discrete units such as capsules, cachets or tablets, wherein each preferably contains a predetermined amount of the one or more curcuminoids and/or vitamin B6; as a powder or granules; as a solution or suspension in an aqueous or non-aqueous liquid; or as an oil-in-water liquid emulsion or a water-in-oil liquid emulsion. In other embodiments, the composition is presented as a bolus, electuary, or paste, etc.

Preferred unit dosage formulations are those containing a daily dose or unit, daily subdose, as herein above-recited, or an appropriate fraction thereof, of an agent.

It should be understood that in addition to the ingredients particularly mentioned above, the compositions may include other agents conventional in the art having regard to the route of administration in question. For example, compositions suitable for oral administration may include such further agents as sweeteners, thickeners and flavoring agents. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

Various delivery systems are known and can be used to administer compositions described herein, e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the compositions of the disclosure locally to the area in need of treatment; this may be achieved by, for example, and not by way of limitation, local infusion during surgery, injection, or by means of a catheter.

Therapeutic amounts are empirically determined and vary with the pathology being treated, the subject being treated and the efficacy and toxicity of the agent. When delivered to an animal, the method is useful to further confirm efficacy of the agent.

It also is intended that the compositions and methods of this disclosure be combined with other suitable compositions and therapies. For example, the compositions described herein may co-administered with one or more additional agents suitable for the prevention of the inflammatory disease (e.g., colorectal cancer).

The one or more curcuminoids and the vitamin B6 may be administered to the subject at any desired frequency. For example, the one or more curcuminoids may be administered to the subject more than once per day (e.g. twice per day, three times per day, four times per day, and the like), once per day, once every other day, once a week, and the like. The one or more curcuminoids and the vitamin B6 may be provided to the subject for any desired duration. For example, the one or more curcuminoids and the vitamin 136 may be administered to the subject for at least one week, at least two weeks, at least three weeks, at least one month, at least two months, at least three months, at least six months, at least one year, at least two years, at least three years, at least four years, at least five years, at least ten years, at least twenty years, or for the lifetime of the subject.

The one or more curcuminoids and vitamin B6 may be provided to the subject to prevent any suitable inflammatory disease in the subject. In particular embodiments, the inflammatory disease is cancer. In some embodiments, the cancer may be selected from colorectal cancer, colon cancer, breast cancer, pancreatic cancer, and uterine cancer. For example, the inflammatory disease may be colorectal cancer.

In some embodiments, the inflammatory disease is a disease or condition known to affect the bowel, for example inflammatory bowel disease such as ulcerative colitis or Crohn's disease.

In other embodiments, the inflammatory disease is an inflammatory condition that affects areas outside of the bowel, such as, for example, arthritis, insulin resistance and diabetes, or non-alcoholic fatty liver disease.

In some embodiments, preventing the inflammatory disease reduces the likelihood of the inflammatory disease from occurring (e.g. reduces the risk of developing the inflammatory disease) in a subject not presently experiencing or afflicted with the inflammatory disease. For example, the method of preventing colorectal cancer may reduce the likelihood of colorectal cancer occurring in a subject not presently experiencing or afflicted with colorectal cancer.

Virtually all colorectal cancers arise from pre-cancerous (adenomatous) polyps, which are not inherently dangerous by themselves other than their propensity for developing into cancer. Therefore, the subject not presently afflicted with colorectal cancer may have pre-cancerous polyps that are not yet considered cancerous. Alternatively, the subject not presently afflicted with colorectal cancer may not have pre-cancerous polyps. In some embodiments, prevention of colorectal cancers may include preventing the formation of pre-cancerous polyps in the subject.

In some embodiments, preventing the inflammatory disease delays the onset of the inflammatory disease in a subject not presently experiencing or afflicted with the inflammatory disease. For example, preventing colorectal cancer may reduce the onset of colorectal cancer in the subject. The terms do not necessarily indicate complete or absolute prevention.

The subject may be at risk of developing the inflammatory disease. For example, the subject may be at risk of developing colorectal cancer. The risk may be the result of a clinical finding selected from a family history of colorectal cancer, a prior history of colorectal cancer, a finding of a polyp or precancerous lesion during colonoscopy, and/or a finding of a molecular marker associated with colorectal cancer (See e.g., Alquist, GASTROENTEROLOGY 2009; 136:2068-2073; herein incorporated by reference in its entirety).

In some embodiments, the subject is obese or overweight. The subject may be identified as obese or overweight based upon the body mass index (BMI) as known in the art. In particular embodiments, the subject is an obese or overweight person at risk of developing colorectal cancer. In other embodiments, the subject is not obese or overweight.

### EXPERIMENTAL

The following examples are provided in order to demonstrate and further illustrate certain preferred embodiments and aspects of the present disclosure and are not to be construed as limiting the scope thereof.

### Example 1

### Introduction

Colorectal cancer (CRC) is a major public health issue in the U.S., with over 145,000 new cases will be diagnosed in the U.S. in 2019, and over 51,000 individuals will die from the disease. CRC is the third most common cancer in each gender and the second most common cause of cancer death in the U.S. Epidemiological studies have demonstrated that obesity is a prominent risk factor for CRC in both sexes, with a 1.5- to 2-fold increase in men, and a 1.2- to 1.5-fold increase in women. Low-grade, chronic inflammation in the colons produced by obesity may be an essential factor in mediating the pro-carcinogenic effects of obesity in colorectal tumorigenesis. Biochemical inflammation may be elevated in the colons of obese laboratory rodents and humans as compared with lean controls. Incremental elevations in tumor necrosis factor (TNF)-α and interleukin (IL)-6 in the colon associated with increasing BM, and activation of several pro-cancerous signaling pathways, such as NF-κB and ERK 1/2 in obese subjects compared to lean controls is also possible. Even a modest degree of adiposity induced by high-fat diet (HFD) could significantly elevate the colonic levels of IL-6, IL-1β and TNF-α, and promote the formation of colorectal tumors in mice.

Inflammatory cytokines are known to activate the PI3K pathway, which promotes a variety of cellular processes including cell growth, cell survival and proliferation. PI3K-mediated phosphorylation has a wide range of downstream targets, including NF-κB, which is a transcription factor that up-regulates cell survival signaling pathways. NF-κB may play a role in the initiation and progression of the majority of colorectal cancers, as NF-κB exerts a variety of pro-tumorigenic functions. Activation of PI3K also stimulates the expression of pro-inflammatory cytokines such as TNF-α, IL-1β, IL-6 and TL-8, which in turn activate NFκB, creating a positive feedback loop. Wnt signaling, the over-activation of which is a fundamental early step in over 85% of CRCs, is similarly up-regulated by the PI3K pathway. This occurs via phosphorylation (=inactivation) of one of its negative regulatory elements, glycogen synthase kinase 3 beta (GSK3β), Which in turn stabilizes the ultimate effector protein of canonical Wnt signaling, β-catenin. Transcriptomic analyses of colonic mucosa from human subjects have demonstrated that NF-κB and other pro-inflammatory/carcinogenic pathways are activated in obese individuals, underscoring the clinical relevance of this phenomenon.

This example demonstrates that the combination of curcumin and vitamin B6 is highly efficacious for the prevention of colorectal cancer compared to the effects of curcumin or vitamin B6 alone.

### Methods

Chemicals, reagents and antibodies: AOM was purchased from Sigma-Aldrich (St. Louis, Mo., USA). CUR (as Curcumin C3 Complex, a mixture of curcuminoids: 79.5% curcumin, 17.9% demethoxycurcumin and 2.8% bisdemethoxycurcumin) was a gift from the Sabinsa Corporation (East Windsor, N.J., USA). Customized rodent diets were purchased from Research Diets (New Brunswick, N.J., USA), and diet composition is shown in Table 1. The V-PLEX Proinflammatory Panel 1 (mouse) Kit and Tris Lysis Buffer containing 150 mM NaCl, 20 mM Tris, pH 7.5, 1 mM EDTA, 1 mM EGTA, 1% Triton X-100 were obtained from Meso Scale Discovery (Rockville, Md., USA). Protease and phosphatase inhibitors were obtained from Boston Bio Products (Ashland, Mass., USA). The Nuclear Extraction Kit was from Abcam (Cambridge, Mass., USA). Protein concentrations were determined using the BCA method (Pierce, Rockford, Ill., USA). SuperSignal™ West Femto Maximum Sensitivity Substrate and CL-XPosure™ Film were from Thermo Fisher Scientific (Waltham, Mass., USA). Antibodies for phospho-PI3K (p85 (Tyr458)/p55 (Tyr199), #4228S), PI3K (#4257S), phospho-NF-κB p65 (Ser536, #3033S), β-catenin (#8480S) and Lamin B1 (#134355) were from Cell Signaling Technology (Beverly, Mass., USA). All the antibodies used in western blot were at 1:1000 dilutions.

TABLE 1
Rodent diet composition, final body weight and body composition of mice.
Data are shown as the mean ± SE. Different superscript letters in each column indicate
significant differences between the five groups (p < 0.05) by one-way analysis of variance
(ANOVA) and Tukey's post-hoc test.

| | Kcal % | Pyridoxine | CUR | Final body | | |
|---|---|---|---|---|---|---|
| Group | from fat | HCL (B6) | (w/w in diet) | weight (g) | % Fat Mass | % Lean Mass |
| LFD | 10% | 6 mg/kg | none | 32.03 ± 0.55 ^{a} | 27 ± 0.79 ^{a} | 69 ± 0.75 ^{a} |
| HFD | 60% | 6 mg/kg | none | 38.35 ± 0.65 ^{b} | 34 ± 0.55 ^{b} | 62 ± 0.35 ^{b} |
| CUR | 60% | 6 mg/kg | 0.2% | 35.17 ± 0.77 ^{ab} | 31 ± 0.71 ^{ab} | 66 ± 0.70 ^{ab} |
| B6 | 60% | 24 mg/kg | none | 36.11 ± 0.69 ^{ab} | 32 ± 1.37 ^{ab} | 64 ± 1.29 ^{ab} |
| C + B | 60% | 24 mg/kg | 0.2% | 37.06 ± 0.79 ^{ab} | 33 ± 0.72 ^{ab} | 63 ± 0.67 ^{ab} |

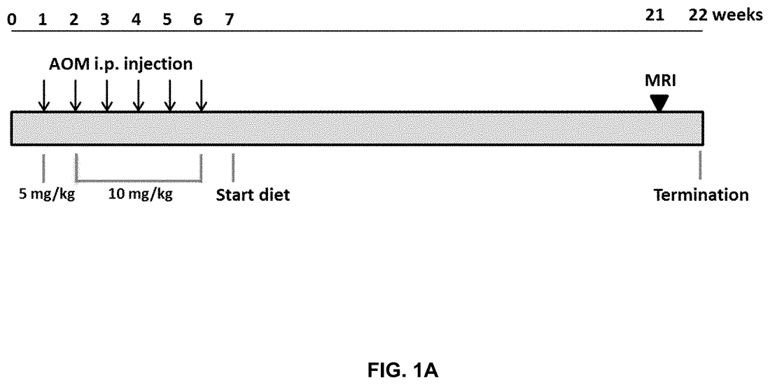

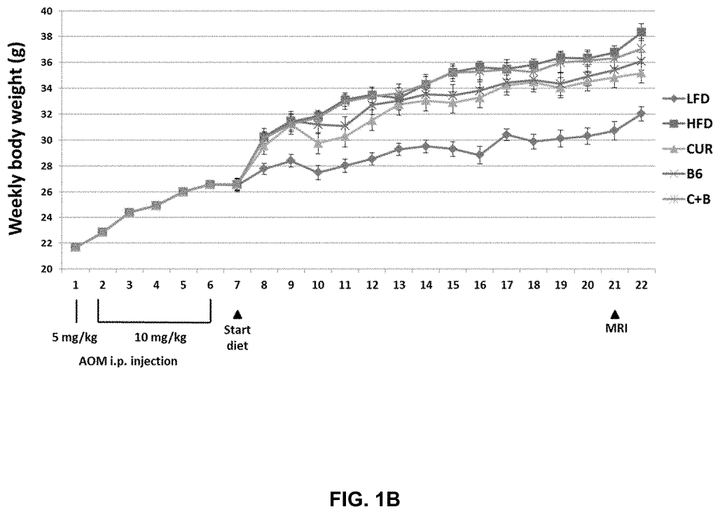

Animals, diet and dosage information: The animal protocol was approved by the Institutional Animal Care and Use Committee of Tufts University (42015-155). Male FVB mice (4 weeks old) were obtained from the Jackson Laboratory (Bar Harbor, Me., USA), and were given free access to food (AIN-93G) and water for one week. The high-fat diet (HFD, D12450B) and low-fat diet (LFD, D12492) contained 60% and 10% of their calories as lard, respectively (Research Diets, diet details: Supplementary Tables 1, 2). As shown in FIG. 1A, starting from the second week after arrival, mice were given 6 weekly injections of AOM (5 mg/kg i.p. at the first injection, and 10 mg/kg i.p at the second to the sixth injections). One week after the sixth AOM injection, mice were randomized to receive 1 of 5 experimental diets: 1) LFD control, 2) RFD control, 3) HFD containing 0.2% CUR (wt %), 4) HFD containing B6 (2.4 mg/kg, at 4-times the basal requirement, as pyridoxine HO), and 5) HFD containing both agents (CUR/136). There were 21 mice in each group. Body composition was determined by Echo-900 MRI (Houston, Tex., USA) at 21 weeks after arrival. All mice were sacrificed by isoflurane asphyxiation at 22 weeks after arrival. The liver and spleen were collected and weighed.

The dose of CUR (0.2 wt %) used in this study is equivalent to approximately 1 g/day for human dietary intake in a 60 kg adult based on equivalent surface area dosage conversion method⁽³⁰⁾. The dose of B6 used is at 4-times the basal requirement (supplemented level) for mice. The doses of CUR and B6 are a reasonably achievable in humans, and a prior clinical study in patients with ulcerative colitis showed that CUR at similar dose (1 g after breakfast and 1 g after the evening meal) exerted significant anti-inflammatory efficacy and was well-tolerated⁽²¹⁾. The colons were excised, opened longitudinally, rinsed with ice cold PBS containing protease and phosphatase inhibitors (pH 7.4) and carefully inspected under a dissection microscope. The number and size of tumors in the colons were recorded. The sizes of tumors were determined by the following formula: tumor volume (mm³)=L×W×[(L+W)/2], where L is the length and W is the width of the tumors. Tumors and one 2-3 mm length piece of mid-colon from each mouse were fixed in 10% buffered formalin (pH 7.4) for 24 h for further histopathologic and immunohistochemical (IHC) analysis. The opened colon, spread out on a glass plate sitting atop crushed ice, was then gently scraped using glass microscope slides and the mucosa thus obtained was stored at −80° C. for future analyses. The pathology of the colonic tumors was characterized by a rodent histopathologist at the Harvard Rodent Histopathology Core (Boston, Mass., USA) who was blinded to the diet groups, and the tumors were graded as either non-neoplastic, adenomas or invasive cancers. The tumor data reported below includes only those confirmed to be either adenomas or cancers.

Determination of pro-inflammatory cytokines in plasma and calprotectin in feces: Plasma samples were then used for determination IL-1β, IL-6, and TNT-α concentrations by V-PLEX Proinflammatory Panel 1 (mouse) Kit on a chemiluminescence platform (MesoScale Discovery (MSD), Rockville, Md., USA) according to the manufacturer's instructions. For fecal calprotectin measures, frozen fecal samples obtained in the colon upon euthanasia were lysed in ice-cold PBS containing 1% BSA, 0.05% Tween20 and protease inhibitor. Protein concentrations were determined using the BCA method. Fecal calprotectin levels were measured by Mouse S100A8/S100A9 Heterodimer DuoSet ELISA according to manufacturer's instructions (R&D Systems, Minneapolis, Minn.) and normalized by protein concentrations.

Western blot analyses: The nuclear and cytoplasmic fractions were prepared using the Nuclear Extraction Kit (Abeam) according to the manufacturer's instructions and subjected to Western blot analysis. Protein concentrations were determined using the BCA method. Briefly, equal amount of proteins (50 μg) were resolved over 4-12% SDS-polyacrylamide gel electrophoresis and transferred to nitrocellulose membranes. After blocking, proteins of interest were probed using different antibodies at the manufacturer's recommended concentrations and then visualized using SuperSignal™ West Femto Maximum Sensitivity Substrate (Thermo Fisher Scientific) and CL-XPosure™ Film (Thermo Fisher Scientific) according to the manufacturer's instructions. Band intensity was normalized to Lamin B1 (nuclear fraction) and measured by Quantity One 1-D Analysis Software (Bio-Rad, Hercules, Calif., USA).

Statistical analyses: Data are presented as means±standard error (SE) for the indicated number of independently performed experiments. All statistical analyses were performed using SAS Version 9.3 (SAS Institute, Cary, N.C., USA). One-way analysis of variance (ANOVA) with Tukey's test was used for the comparison of differences among groups. The tumor multiplicity was square root transformed in order to satisfy distributional assumptions for the ANOVA model. The tumor incidence was statistically calculated using the chi-square test. Due to the multiplicity of signaling pathways that were examined multiple comparisons with the Hochberg method were employed to control the False Discovery Rate. A p value of <0.05 was considered statistically significant.

### Results and Discussion

Development of HFD-induced obesity in FVB mice: No differences in food intake, behavior, or appearance were observed among the five groups over the entire course of the experiment. One mouse died after the second AOM injection, and one week after being randomized into different groups, one mouse in the HFD group died prematurely: the causes of death were not evident. Body weight was monitored weekly (FIG. 1B). Mice consuming the HFD without additives (Group 2) quickly achieved a significant greater body weight starting from one week after switching to RFD, compared to the LED mice (Group 1) (p<0.01), and the significant differences between TIED- and LED-mice were observed throughout the entire course of the experiment. The final body weight of mice consuming HFD was 38.35±0.65 g, while mice on the LFD were 32.53±0.55 g (Table 1), which is 18% difference (p<0.01). MRI result showed that the proportion of the body comprised of fat mass in the HFD mice was also 26% greater (34% vs. 27%, p<0.01). Altogether, 60% RFD successfully induced obesity in FVB mice.

All three intervention groups (Group 3-5) developed a numerically—but not statistically significant—lower fat mass compared to the HFD-fed obese mice. Supplementation of CUR (Group 3) or B6 (Group 4) resulted in a reduction in final body weight, in comparison to the obese controls (Group 2) (p<0.03). Interestingly, the combination of CUR/B6 (Group 5) only caused a non-significant lower final body weight, an effect likely due to the much smaller tumor multiplicity and burden possessed by this group (Table 2).

TABLE 2
Colonic tumor data of HFD-fed, AOM-treated FVB mice. Data are
shown as the mean ± SE. Different superscript letters in each column
indicate significant differences between the five groups (p < 0.05) by
one-way analysis of variance (ANOVA) and Tukey's post-hoc test.
The tumor multiplicity was square root transformed in order to satisfy
distributional assumptions for the ANOVA model. The tumor incidence
was compared between groups using the chi-square test.

| | | Tumor burden | |
|---|---|---|---|
| | Tumor multiplicity | (Total volume of tumors | Tumor |
| Group | (Tumors per mouse) | per mouse, mm³) | incidence |
| LFD | 0.64 ± 0.18^{a} | 18.36 ± 7.14^{ab} | 45% |
| HFD | 1.38 ± 0.21^{b} | 36.80 ± 9.26^{a} | 81% |
| CUR | 0.81 ± 0.19^{ab} | 25.35 ± 14.29^{ab} | 52% |
| B6 | 0.95 ± 0.22^{ab} | 17.19 ± 7.38^{ab} | 59% |
| C + B | 0.50 ± 0.13^{a} | 8.18 ± 3.60^{b} | 45% |

The combination regimen of CUR/B6 diminishes colon tumorigenesis in AOM-treated obese mice: FVB mice may be susceptible to AOM-induced colorectal tumorigenesis. As shown in Table 2, the HFD-induced obesity, in conjunction with six injections of AOM, resulted in a 2.2-fold increase in tumor multiplicity compared to the lean mice (p<0.05). The obese mice had 1.38±0.21 tumors each, whereas lean mice had 0.64±0.18 tumors each. Obese control group also showed a 2.0- and 1.8-fold greater tumor burden and incidence compared to the lean control, although the changes did not reach a statistical significance. Altogether, the obesity generated by 60% HFD promoted the formation of colonic tumors in AOM-treated FVB mice.

Supplementation of CUR/136 significant suppressed CRC development in mice, as evidenced by drastic 64% and 78% reduction in tumor multiplicity and burden, respectively, compared to the obese control (p<0.03). In contrast, neither CUR nor B6 alone was sufficient to suppress colorectal tumorigenesis. Tumor incidence was not significantly different between the five groups, although the combination resulted in a level as low as that was seen in the lean control. Thus, the combination regimen of CUR/B6 substantially attenuated two of the three metrics of CRC, and the singular intervention was largely ineffective in suppressing any tumor metrics enhanced by obesity.

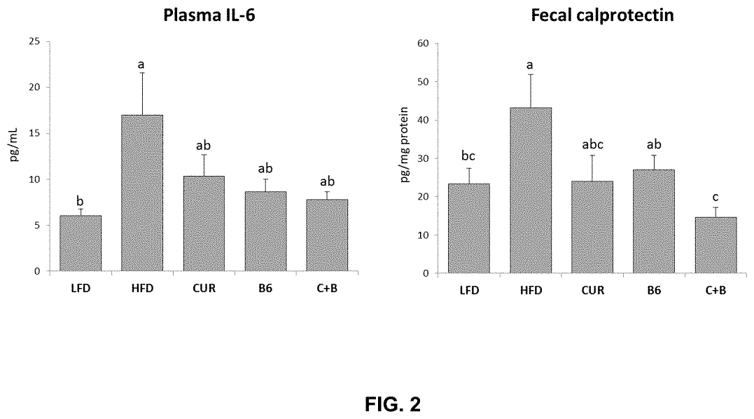

Suppression of obesity-induced inflammation by the combination regimen: Levels of critical pro-inflammatory cytokines in plasma and calprotectin in feces were measured to determine whether the obesity-generated by HFD essentially induced chronic inflammation in AOM-treated. FVB mice. Obesity caused a 2.81-fold elevation in IL-6 level in plasma compared to the level observed in the lean control group (p<0.02), as shown in FIG. 2. Supplementation of CUR, B6 or their combination only resulted in a non-significant reduction in plasma IL-6 level, although the combination showed a tendency to attenuate this obesity-induced systemic inflammation (p=0.07). There were no significant differences in the mean plasma concentrations of IL 1β and TNF-α between the five groups (data not shown).

Level of calprotectin, a neutrophil-driven calcium-binding protein, in feces is used as a marker of intestinal inflammation in clinical and pre-clinical settings. In the present study, obesity led to a 1.85-fold increase the concentration of calprotectin in feces in comparison to LFD-fed mice (p<0.05). Combination of CUR/B6 greatly reduced fecal calprotectin level by 66%, compared to the obese control group (p<0.01). In contrast, singular supplementation of CUR or B6 did not cause a significant suppression. Taken together, the combination treatment has an apparent advantage over either agent alone in suppressing obesity-induced inflammation.

### CUR/B6 Combination Regimen Modulates Multiple Pro-Carcinogenic Signaling Pathways in the Colon

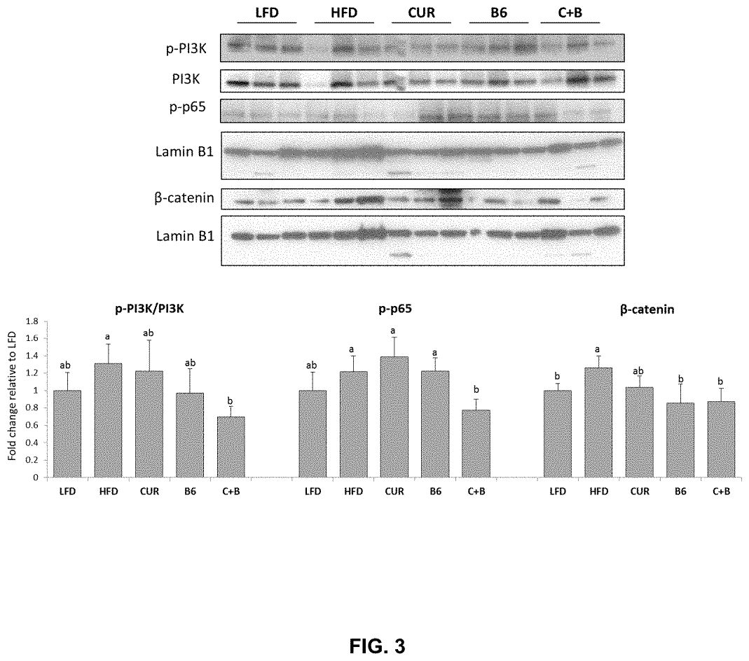

Wnt: It is well accepted that overly-activated Wnt signaling is an early event in 90% of human CRC. In mice, diet-induced obesity elevates colonic cytokines and that these elevations are accompanied by increased Writ signaling. Activated Wnt signaling can occur through phosphorylation (=inactivation) of GSK3β, a negative regulatory element, which in turn causes protein stabilization and facilitation of nuclear localization of the major effector of canonical Wnt signaling, β-catenin. As shown in FIG. 3, the obese mice exhibited a significantly higher nuclear expression level of β-catenin compared to lean control mice (p<0.03), indicating that upregulation of Win signaling was involved in the obesity-promoted CRC in AOM-treated FVB mice. Both B6 alone and the combination of CUR and B6 led to a reduced level of nuclear β-catenin, in comparison to the obese control (p<0.03), while CUR alone did not down-regulated that.
PI3K: Phosphorylation of the PI3K is the essential step in its activation and the upregulation of downstream signaling, and the phosphorylated protein/total protein ratio is widely used as markers of activation of PI3K pathway⁽³⁷⁾. The combination of CUR/B6 significantly diminished the activation of PI3K signaling induced by obesity by 47% (p<0.05). In contrast, CUR and B6 alone did not cause a significant suppression. Altogether, CUR/B6 co-administration produced potent inhibitory effects on the PI3K signaling pathways to a degree that was not seen with either agent alone.
NF-κB: Transcription factor p65 is a functional subunit of NF-κB. When dissociated from its cytoplasmic inhibitor IκB, p65 can translocate into the cell nucleus and exert its transcriptional activities, including modulation of immune responses, inhibition of apoptosis, promotion of cellular proliferation and angiogenesis. Phosphorylation of at Ser536 in the transactivation domain of NF-κB (p65) by the catalytic subunit of protein kinase A is an important modification that further enhances its transcriptional activity. Therefore, protein level of phosphor-p65 was determined in the nuclear fraction of the colonic mucosa. The combination of CUR and B6 resulted in a substantial decrease in phosphor-p65 compared to the obese control group (p<0.01), which was not observed in singular treatment groups. Overall, the combination of CUR/B6 exerted the strongest effect in suppressing obesity-activated NF-κB signaling, and the result is in accordance with that of the upstream effector PI3K.

All publications, patents and patent applications mentioned in the above specification are herein incorporated by reference in their entirety. Although the disclosure has been described in connection with specific embodiments, it should be understood that the disclosure as claimed should not be unduly limited to such specific embodiments. Indeed, various modifications and variations of the described compositions and methods of the disclosure will be apparent to those of ordinary skill in the art and are intended to be within the scope of the following claims.

1. A method of preventing an inflammatory disease in a subject comprising providing to the subject one or more curcuminoids and vitamin B6.

2. The method of claim 1, wherein the one or more curcuminoids and the vitamin B6 are provided to the subject jointly in a single composition.

3. The method of claim 1, wherein the one or more curcuminoids and the vitamin B6 are provided to the subject separately in two or more compositions.

4. The method of claim 1, wherein the one or more curcuminoids are selected from curcumin, demethoxycurcumin, bisdemethoxycurcumin, and combinations thereof.

5. The method of claim 4, wherein the one or more curcuminoids comprise 1-100% curcumin, 0-99% demethoxycurcumin, and 0-99% bisdemethoxycurcumin.

6. The method of claim 5, wherein the one or more curcuminoids comprise 60-100% curcumin, 1-50% demethoxycurcumin, and 1-30% bisdemethoxycurcumin.

7. The method of claim 6, wherein the one or more curcuminoids comprise 70-90% curcumin, 10-20% demethoxycurcumin, and 1-10% bisdemethoxycurcumin.

8. The method of claim 1, wherein the vitamin B6 is provided as pyridoxine or as a salt thereof.

9. The method of claim 1, wherein the inflammatory disease is selected from colorectal cancer, colon cancer, irritable bowel syndrome, ulcerative colitis, and Crohn's disease.

10. The method of claim 9, wherein the inflammatory disease is colorectal cancer or pre-cancerous polyps.

11. The method of claim 1 wherein the subject is obese or overweight.

12. The method of claim 1, wherein the subject is not obese or overweight.

13. The method of claim 1, wherein the subject is at risk of developing colorectal cancer.

14. The method of claim 13, wherein the risk is the result of a clinical finding selected from a family history of colorectal cancer, a prior history of colorectal cancer, a finding of a polyp or precancerous lesion during colonoscopy, and a finding of a molecular marker associated with colorectal cancer.

15. The method of claim 1, wherein the composition is provided orally to the subject.

16. A composition comprising one or more curcuminoids and vitamin B6.

17. The composition of claim 16, wherein the one or more curcuminoids are selected from curcumin, demethoxycurcumin, bisdemethoxycurcumin, and combinations thereof.

18. (canceled)

19. The composition of claim 18, wherein the one or more curcuminoids comprise 60-100% curcumin, 1-50% demethoxycurcumin, and 1-30% bisdemethoxycurcumin.

20. The composition of claim 19, wherein the one or more curcuminoids comprise 70-90% curcumin, 10-20% demethoxycurcumin, and 1-10% bisdemethoxycurcumin.

21. The composition of claim 16, wherein the vitamin B6 is provided as pyridoxine or as a salt thereof.

22-30. (canceled)
